(19) United States
(12) Patent Application Publication
(10) Pub. No.: US 2024/0346718 A1
(43) Pub. Date: Oct. 17, 2024

(54) GENERATION OF ARTIFICIAL CONTRAST-ENHANCED COMPUTED TOMOGRAPHY IMAGES

(71) Applicant: Bayer Akiengesellschaft, Leverkusen (DE)

(72) Inventors: Gregor JOST, Leverkusen (DE); Felix Karl KREIS, Leverkusen (DE); Johannes HÕHNE, Leverkusen (DE); Gunnar SCHÛTZ, Leverkusen (DE); Hubertus PIETSCH, Leverkusen (DE)

(73) Assignee: Bayer Aktiengesellschaft, Leverkusen (DE)

(21) Appl. No.: 18/633,989

(22) Filed: Apr. 12, 2024

(30) Foreign Application Priority Data
Apr. 13, 2023 (EP) 23167656.0

(51) Int. Cl. G06T 11/00

(52) U.S. Cl. CPC G06T 11/006; G06T 2211/408; G06T 2211/461

(57) ABSTRACT

Disclosed herein are systems and methods for generating artificial contrast-enhanced computed tomography (CT) images. An exemplary computer-implemented method involves receiving representations of an examination region of an examination object after administration of a contrast agent. The representations result from CT examination of the examination region at different X-ray energies. The method involves generating a representation of the contrast agent signals on the basis of the received representations (e.g., a signal intensity distribution brought about by the contrast agent in the examination region). The method involves generating a synthetic representation of the examination region comprising an α-fold addition of the representation of the contrast agent signals to one of the received representations or to a virtual non-contrast agent representation of the examination region. α is a negative or positive real number. The method involves outputting, storing, and/or transmitting the synthetic representation to a separate computer system.

### CROSS-REFERENCE TO RELATED APPLICATIONS

This application claims priority to European Application No. 23167656.0, filed on Apr. 13, 2023, the entire content of which is hereby incorporated by reference in its entirety.

### TECHNICAL FIELD

The present disclosure is concerned with the technical field of generation of artificial contrast-enhanced computed tomography images.

### INTRODUCTION

WO2019/074938A1 discloses a method for reducing the amount of contrast agent in the generation of radiological images with the aid of an artificial neural network.

In the disclosed method, a training data set is created in a first step. The training data set comprises for each person of a multiplicity of persons i) a native radiological image (zero-contrast image), ii) a radiological image after administration of a small amount of contrast agent (low-contrast image) and iii) a radiological image after administration of a standard amount of contrast agent (full-contrast image).

In a second step, an artificial neural network is trained to predict for each person of the training data set, on the basis of the native image and the image after administration of a low amount of contrast agent, an artificial radiological image showing an acquisition region after administration of the standard amount of contrast agent. The measured radiological image after administration of a standard amount of contrast agent is used in each case as reference (ground truth) in the training.

In a third step, the trained artificial neural network can be used to predict for a new person, on the basis of a native image and of a radiological image after the administration of a low amount of contrast agent, an artificial radiological image which shows the acquired region as it would look if a standard amount of contrast agent had been administered.

The method disclosed in WO2019/074938A1 has disadvantages.

For instance, training data are required for training the artificial neural network. A large number of radiological examinations need to be carried out on a large number of persons and the training data need to be generated in order to be able to train the network.

The artificial neural network disclosed in WO2019/074938A1 is trained to predict a radiological image after administration of a standard amount of a contrast agent. The artificial neural network is not configured and not trained to predict a radiological image after administration of an amount lower or higher than the standard amount of contrast agent. The method described in WO2019/074938A1 can in principle be trained to predict a radiological image after administration of an amount of contrast agent different than the standard amount. However, this requires further training data and further training.

Besides reducing the amount of contrast agent, there may be other motives for boosting the signal intensities brought about by a contrast agent in a computed tomography (CT) image. If, for example, a contrast agent is used in a computed tomography examination method that is intended for a magnetic resonance imaging (MRI) examination, such an MRI contrast agent usually results, in a CT image, in lower signal enhancement compared to a CT contrast agent. One motive for boosting the signal intensity brought about by contrast agents may therefore be the desire to boost the low signal intensity brought about by an MRI contrast agent in a CT image. The use of an MRI contrast agent in a computed tomography examination is described for example in J. Thomas et al.: Gadoxetate disodium enhanced spectral dual-energy CT for evaluation of cholangiocarcinoma: Preliminary data, Annals of Medicine and Surgery, 2016, 6. 10.1016/j.amsu.2016.01.001.

It would also be desirable to be able to generate CT images with variable contrast enhancement without needing to generate training data for each individual contrast enhancement and without the need to train an artificial neural network. It would additionally be desirable to be able to generate CT images with variable contrast enhancement using a trackable deterministic process to generate the variable contrast enhancement. This facilitates the approval and use of a corresponding medical procedure, while minimizing false negative and false positive results. Machine learning methods employ statistical models, the generalizability of which is limited because they are usually based on a limited selection of training data. It would additionally be desirable to be able to generate CT images with variable contrast enhancement using a wide variety of contrast agents. It would additionally be desirable to be able to use the method for generating CT images with variable contrast enhancement using a wide variety of different contrast agents irrespective of their physical, chemical, physiological or other properties.

### BRIEF SUMMARY

These and other objects are achieved by the subject-matter of the independent claims. Preferred embodiments of the present disclosure are found in the dependent claims, in the present description and in the drawings.

The present disclosure thus provides in a first aspect a computer-implemented method for generating a synthetic contrast-enhanced CT image, comprising the steps of:
receiving and/or generating at least two representations of an examination region of an examination object, where the at least two representations represent the examination region after administration of a contrast agent, where the at least two representations are the result of a computed tomography examination of the examination region at different X-ray energies,
generating a representation of the contrast agent signals on the basis of the at least two received representations, where the representation of the contrast agent signals represents a signal intensity distribution brought about by the contrast agent in the examination region,
generating a synthetic representation of the examination region, where the generation of the synthetic representation comprises an α-fold addition of the representation of the contrast agent signals to one of the at least two received representations or to a virtual non-contrast agent representation of the examination region, where α is a negative or positive real number,
outputting and/or storing the synthetic representation and/or transmitting the synthetic representation to a separate computer system.

The present disclosure further provides a computer system comprising:
a processor; and
a memory that stores an application program configured to perform an operation when executed by the processor, said operation comprising the steps of:
receiving and/or generating at least two representations of an examination region of an examination object, where the at least two representations represent the examination region after administration of a contrast agent, where the at least two representations are the result of a computed tomography examination of the examination region at different X-ray energies,
generating a representation of the contrast agent signals on the basis of the at least two received representations, where the representation of the contrast agent signals represents a signal intensity distribution brought about by the contrast agent in the examination region,
generating a synthetic representation of the examination region, where the generation of the synthetic representation comprises an α-fold addition of the representation of the contrast agent signals to one of the at least two received representations or to a virtual non-contrast agent representation of the examination region, where α is a negative or positive real number,
outputting and/or storing the synthetic representation and/or transmitting the synthetic representation to a separate computer system.

The present disclosure further provides a computer program that can be loaded into a working memory of a computer system, where it causes the computer system to execute the following steps:
receiving and/or generating at least two representations of an examination region of an examination object, where the at least two representations represent the examination region after administration of a contrast agent, where the at least two representations are the result of a computed tomography examination of the examination region at different X-ray energies,
generating a representation of the contrast agent signals on the basis of the at least two received representations, where the representation of the contrast agent signals represents a signal intensity distribution brought about by the contrast agent in the examination region,
generating a synthetic representation of the examination region, where the generation of the synthetic representation comprises an α-fold addition of the representation of the contrast agent signals to one of the at least two received representations or to a virtual non-contrast agent representation of the examination region, where α is a negative or positive real number,
outputting and/or storing the synthetic representation and/or transmitting the synthetic representation to a separate computer system.

The present disclosure further provides for the use of a contrast agent in a CT examination method, wherein the CT examination method comprises the steps of:
receiving and/or generating at least two representations of an examination region of an examination object, where the at least two representations represent the examination region after administration of the contrast agent, where the at least two representations are the result of a computed tomography examination of the examination region at different X-ray energies,
generating a representation of the contrast agent signals on the basis of the at least two received representations, where the representation of the contrast agent signals represents a signal intensity distribution brought about by the contrast agent in the examination region,
generating a synthetic representation of the examination region, where the generation of the synthetic representation comprises an α-fold addition of the representation of the contrast agent signals to one of the at least two received representations or to a virtual non-contrast agent representation of the examination region, where α is a negative or positive real number,
outputting and/or storing the synthetic representation and/or transmitting the synthetic representation to a separate computer system.

The present disclosure further provides a contrast agent for use in a CT examination method, wherein the CT examination method comprises the steps of:
receiving and/or generating at least two representations of an examination region of an examination object, where the at least two representations represent the examination region after administration of the contrast agent, where the at least two representations are the result of a computed tomography examination of the examination region at different X-ray energies,
generating a representation of the contrast agent signals on the basis of the at least two received representations, where the representation of the contrast agent signals represents a signal intensity distribution brought about by the contrast agent in the examination region,
generating a synthetic representation of the examination region, where the generation of the synthetic representation comprises an α-fold addition of the representation of the contrast agent signals to one of the at least two received representations or to a virtual non-contrast agent representation of the examination region, where α is a negative or positive real number,
outputting and/or storing the synthetic representation and/or transmitting the synthetic representation to a separate computer system.

The present disclosure further provides a kit comprising a contrast agent and a non-volatile storage medium comprising a computer program that, when loaded into a working memory of a computer system, causes the computer system to execute the following steps:
receiving and/or generating at least two representations of an examination region of an examination object, where the at least two representations represent the examination region after administration of a contrast agent, where the at least two representations are the result of a computed tomography examination of the examination region at different X-ray energies,
generating a representation of the contrast agent signals on the basis of the at least two received representations, where the representation of the contrast agent signals represents a signal intensity distribution brought about by the contrast agent in the examination region,
generating a synthetic representation of the examination region, where the generation of the synthetic representation comprises an α-fold addition of the representation of the contrast agent signals to one of the at least two received representations or to a virtual non-contrast agent representation of the examination region, where α is a negative or positive real number,
outputting and/or storing the synthetic representation and/or transmitting the synthetic representation to a separate computer system.

### BRIEF DESCRIPTION OF DRAWINGS

FIGS. 1A-1B show in schematic form an embodiment for the generation of a synthetic contrast-enhanced CT image, in accordance with some embodiments.

FIG. 2 shows in schematic form a further embodiment for the generation of a synthetic contrast-enhanced CT image, in accordance with some embodiments.

FIG. 3 shows in schematic form a further embodiment for the generation of a synthetic contrast-enhanced CT image, in accordance with some embodiments.

FIGS. 4A-4H show examples of frequency-dependent weight functions that can be used for weighting the representation of the contrast agent signals in frequency space, in accordance with some embodiments.

FIG. 5 shows by way of example and in schematic form a computer system according to the present disclosure, in accordance with some embodiments.

FIG. 6 shows by way of example and in schematic form a further embodiment of the computer system according to the present disclosure, in accordance with some embodiments.

FIG. 7 shows by way of example and in schematic form an embodiment of the computer-implemented method for the generation of a synthetic contrast-enhanced CT image, in accordance with some embodiments.

### DETAILED DESCRIPTION

The subject matters of the present disclosure will be more particularly elucidated hereinbelow, without distinguishing between the subject matters (method, computer system, computer program, use, contrast agent for use, kit). Rather, the elucidations that follow are intended to apply by analogy to all subject matters, irrespective of the context (method, computer system, computer program, use, contrast agent for use, kit) in which they occur.

Where steps are stated in an order in the present description or in the claims, this does not necessarily mean that this disclosure is limited to the order stated. Instead, it is conceivable that the steps are also executed in a different order or else in parallel with one another, the exception being when one step builds on another step, thereby making it imperative that the step building on the previous step be executed next (which will however become clear in the individual case). The stated orders thus constitute preferred embodiments.

In certain places the invention will be more particularly elucidated with reference to drawings. The drawings show specific embodiments having specific features and combinations of features, which are intended primarily for illustrative purposes; the invention is not to be understood as being limited to the features and combinations of features shown in the drawings. Furthermore, statements made in the description of the drawings in relation to features and combinations of features are intended to be generally applicable, that is to say transferable to other embodiments too and not limited to the embodiments shown.

The present disclosure describes means by which a synthetic CT image of an examination region is generated on the basis of at least two representations of the examination region of an examination object that are the result of a computed tomography examination of the examination region at different X-ray energies using a contrast agent.

In the synthetic CT image, the signal intensity distribution brought about by the contrast agent can be varied. Contrast enhancements can be achieved that are otherwise achievable, if at all, only by administering an amount of contrast agent that is above the standard amount.

The “standard amount” is normally the amount recommended by the manufacturer and/or distributor of the contrast agent and/or the amount approved by a regulatory authority and/or the amount specified in a package leaflet for the contrast agent.

The “examination object” is usually a living being, preferably a mammal, very particularly preferably a human.

The “examination region” is a part of the examination object, for example an organ or part of an organ or a plurality of organs or another part of the examination object.

For example, the examination region may be a liver, kidney, heart, lung, brain, stomach, bladder, prostate, female breast, intestine or a part thereof or another part of the body of a mammal (for example a human).

In one embodiment, the examination region includes a liver or part of a liver or the examination region is a liver or part of a liver of a mammal, preferably a human.

In a further embodiment, the examination region includes a brain or part of a brain or the examination region is a brain or part of a brain of a mammal, preferably a human.

In a further embodiment, the examination region includes a heart or part of a heart or the examination region is a heart or part of a heart of a mammal, preferably a human.

In a further embodiment, the examination region includes a thorax or part of a thorax or the examination region is a thorax or part of a thorax of a mammal, preferably a human.

In a further embodiment, the examination region includes a stomach or part of a stomach or the examination region is a stomach or part of a stomach of a mammal, preferably a human.

In a further embodiment, the examination region includes a pancreas or part of a pancreas or the examination region is a pancreas or part of a pancreas of a mammal, preferably a human.

In a further embodiment, the examination region includes a kidney or part of a kidney or the examination region is a kidney or part of a kidney of a mammal, preferably a human.

In a further embodiment, the examination region includes one or both lungs or part of a lung of a mammal, preferably a human.

In a further embodiment, the examination region includes a breast or part of a breast or the examination region is a breast or part of a breast of a female mammal, preferably a female human.

In a further embodiment, the examination region includes a prostate or part of a prostate or the examination region is a prostate or part of a prostate of a male mammal, preferably a male human.

The examination region, also referred to as the field of view (FOV), is in particular a volume that is imaged in CT images. The examination region is typically defined by a radiologist, for example on a localizer image. It is of course also possible for the examination region to be alternatively or additionally defined in an automated manner, for example on the basis of a selected protocol.

The examination region is subjected to a computed tomography examination. Computed tomography (CT) is an imaging method based on X-rays. Typically, a rotating X-ray tube revolves around the usually recumbent examination object. The X-rays penetrate the examination region and are attenuated to varying degrees according to the density of the tissue in the different organs. Tissue with high density (for example bone tissue) usually appears as light regions in the images, whereas tissue with low density usually appears dark.

The X-rays can be collected by means of opposing detectors. A computer then calculates cross-sectional images and/or three-dimensional images of the examination region from the signal intensities measured by the detectors.

The computed tomography examination is carried out using a contrast agent. “Contrast agents” are substances or mixtures of substances that improve the depiction of structures and functions of the body in radiological examinations.

The contrast agent may be a CT contrast agent. The CT contrast agent may for example be an iodine-containing compound. Examples of CT contrast agents can be found in the literature (see for example A. S. L. Jascinth et al.: Contrast Agents in computed tomography: A Review, Journal of Applied Dental and Medical Sciences, 2016, vol. 2, issue 2, 143-149; H. Lusic et al.: X-ray-Computed Tomography Contrast Agents, Chem. Rev. 2013, 113, 3, 1641-1666).

The contrast agent may be an MRI contrast agent. In magnetic resonance imaging (MRI), superparamagnetic substances or paramagnetic substances are normally used as contrast agents. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), gadobutrol (Gadovist®), gadopiclenol (Elucirem, Vueway) and gadoxetic acid (Primovist®/Eovist®).

The contrast agents most commonly used in MRI are paramagnetic contrast agents based on gadolinium. These agents are usually administered via an intravenous (i.v.) bolus injection.

From their pattern of distribution in tissue, gadolinium-based contrast agents can be roughly divided into extracellular and intracellular contrast agents.

Extracellular contrast agents refer to low-molecular-weight, water-soluble compounds that, after intravenous administration, are distributed to the blood vessels and the interstitial spaces. After a certain, comparatively short period of circulation in the bloodstream, they are excreted via the kidneys. Extracellular MRI contrast agents include, for example, the gadolinium chelates gadobutrol (Gadovist®), gadoteridol (Prohance®), gadoteric acid (Dotarem®), gadopentetic acid (Magnevist®) and gadodiamide (Omnican®).

Intracellular contrast agents are taken up into the cells of tissues to a certain extent and subsequently excreted. Intracellular MRI contrast agents based on gadoxetic acid are distinguished for example in that they undergo a degree of specific uptake by liver cells (hepatocytes), accumulate in the functional tissue (parenchyma) and enhance contrasts in healthy liver tissue, before being subsequently excreted via the gallbladder into the faeces. Examples of such contrast agents based on gadoxetic acid are described in U.S. Pat. No. 6,039,931A; they are commercially available for example under the trade names Primovist® and Eovist®. A further MRI contrast agent having lower uptake into hepatocytes is gadobenate dimeglumine (Multihance®). Contrast agents that undergo at least a degree of uptake by liver cells are also referred to as hepatobiliary contrast agents. Further hepatobiliary contrast agents are described inter alia in WO2022/194777.

The MRI contrast agent can be an intracellular or extracellular contrast agent. The MRI contrast agent can also be a mixture of more than one (e.g. two) contrast agents.

In one embodiment, the MRI contrast agent is an intracellular, preferably hepatobiliary, contrast agent. In one embodiment, the contrast agent used is a substance or a substance mixture having gadoxetic acid or a salt of gadoxetic acid as contrast-enhancing active substance. For example, it may be the disodium salt of gadoxetic acid (Gd-EOB-DTPA disodium), also known as gadoxetate disodium (GD).

GD is approved at a dose of 0.1 ml/kg body weight (BW) (0.025 mmol/kg BW Gd). The recommended administration of GD comprises an undiluted intravenous bolus injection at a flow rate of about 2 ml/second, followed by flushing of the i.v. cannula with physiological saline.

In a CT examination, MRI contrast agents usually have a lower contrast-enhancing effect than CT contrast agents. However, it can be advantageous to employ an MRI contrast agent in a CT examination. An example is a minimally invasive intervention in the liver of a patient in whom a surgeon is monitoring the procedure by means of a CT scanner. Computed tomography (CT) has the advantage over magnetic resonance imaging that more major surgical interventions are possible in the examination region while generating CT images of an examination region of an examination object. There are only few surgical instruments and surgical devices that are MRI-compatible. Moreover, access to the patient is restricted by the magnets used in MRI. Thus, while a surgeon is performing an operation in the examination region, he/she is able to use CT to visualize the examination region and to monitor the operation.

For example, if a surgeon wishes to perform a procedure in a patient's liver in order for example to carry out a biopsy on a liver lesion or to remove a tumour, the contrast between a liver lesion or tumour and healthy liver tissue will not be as pronounced in a CT image of the liver as it is in an MRI image after administration of a hepatobiliary contrast agent. No CT-specific hepatobiliary contrast agents are currently known and/or approved in CT. The use of an MRI contrast agent, more particularly a hepatobiliary MRI contrast agent, in computed tomography thus combines the possibility of differentiating between healthy and diseased liver tissue and the possibility of carrying out an operation with simultaneous visualization of the liver.

In one embodiment, the contrast agent is gadoxetate disodium.

In one embodiment of the present disclosure, the contrast agent is an agent that includes gadolinium(III) 2-[4,7,10-tris(carboxymethyl)-1,4,7,10-tetrazacyclododec-1-yl]acetic acid (also referred to as gadolinium-DOTA or gadoteric acid).

In a further embodiment, the contrast agent is an agent that includes gadolinium(III) ethoxybenzyldiethylenetriaminepentaacetic acid (Gd-EOB-DTPA); preferably, the contrast agent includes the disodium salt of gadolinium(III) ethoxybenzyldiethylenetriaminepentaacetic acid (also referred to as gadoxetic acid).

In one embodiment of the present disclosure, the contrast agent is an agent that includes gadolinium(III) 2-[3,9-bis[1-carboxylato-4-(2,3-dihydroxypropylamino)-4-oxobutyl]-3,6,9,15-tetrazabicyclo [9.3.1]pentadeca-1(15),11,13-trien-6-yl]-5-(2,3-dihydroxypropylamino)-5-oxopentanoate (also referred to as gadopiclenol; see for example WO2007/042504 and WO2020/030618 and/or WO2022/013454).

In one embodiment of the present disclosure, the contrast agent is an agent that includes dihydrogen [(±)-4-carboxy-5,8,11-tris(carboxymethyl)-1-phenyl-2-oxa-5,8,11-triazatridecan-13-oato(5-)]gadolinate(2-) (also referred to as gadobenic acid).

In one embodiment of the present disclosure, the contrast agent is an agent that includes tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57(10): 629-638; WO2016193190).

In one embodiment of the present disclosure, the contrast agent is an agent that includes a Gd³⁺ complex of a compound of the formula (I)

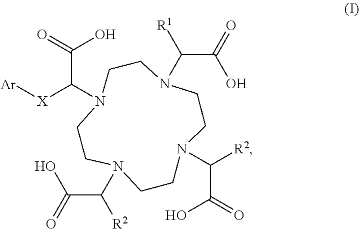

where Ar is a group selected from

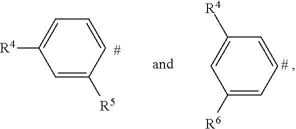

where _{#}is the linkage to X,
X is a group selected from CH₂, (CH₂)₂, (CH₂)₃, (CH₂)₄ and *—(CH₂)₂O—CH₂—^{#},
where * is the linkage to Ar and ^{#}is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom, and
R⁶ is a hydrogen atom, or a stereoisomer, tautomer, hydrate, solvate or salt thereof, or a mixture thereof.

In one embodiment of the present disclosure, the contrast agent is an agent that includes a Gd³⁺ complex of a compound of the formula (II)

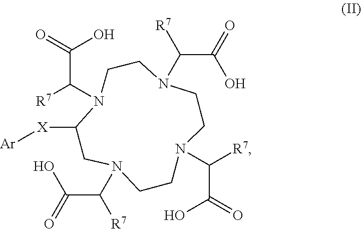

where Ar is a group selected from

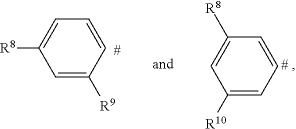

where _{#}is the linkage to X,
X is a group selected from CH₂, (CH₂)₂, (CH₂)₃, (CH₂)₄ and *—(CH₂)₂O—CH₂—^{#}, where * is the linkage to Ar and ^{#}is the linkage to the acetic acid residue,
R⁷ is a hydrogen atom or a group selected from C₁-C₃ alkyl, —CH₂OH, —(CH₂)₂OH and —CH₂OCH₃,
R¹ is a group selected from C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—, and
R⁹ and R¹⁰ are each independently a hydrogen atom, or a stereoisomer, tautomer, hydrate, solvate or salt thereof, or a mixture thereof.

The term “C1-C3 alkyl” denotes a linear or branched, saturated monovalent hydrocarbon group having 1, 2 or 3 carbon atoms, for example methyl, ethyl, n-propyl or isopropyl. The term “C2-C4 alkyl” denotes a linear or branched, saturated monovalent hydrocarbon group having 2, 3 or 4 carbon atoms.

The term “C2-C4 alkoxy” denotes a linear or branched, saturated monovalent group of the formula (C2-C4 alkyl)-O—, in which the term “C2-C4 alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate) (see for example WO2022/194777, example 15).

In one embodiment of the present disclosure, the contrast agent is an agent that includes gadolinium 2,2′,2″-{10-[(1S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium-2,2′,2″-{(2S)-10-(carboxymethyl)-2-[4-(2-ethoxyethoxy)benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium(III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxoethyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

In a first step, at least two representations of an examination region of an examination object are received and/or generated.

The term “receiving” encompasses both the accepting of representations transmitted for example from a separate computer system, and the reading of representations, for example from one or more data memories. The representations may be received from a computed tomography system. The representations may be read from one or more data memories and/or transmitted from a separate computer system.

The term “generating” preferably means that a representation is generated on the basis of another (for example a received) representation or on the basis of a plurality of other (for example received) representations. For example, a received representation may be a representation of the examination region of an examination object in real space. On the basis of this real-space representation it is possible for example to generate a representation of the examination region of the examination object in frequency space through a transform operation (for example a Fourier transform). For example, a received representation may be a representation of the examination region of an examination object in frequency space. On the basis of this frequency-space representation it is possible for example to generate a representation of the examination region of the examination object in real space through a transform operation (for example an inverse Fourier transform). Further options for generating a representation based on one or more other representations are described in this description.

The at least two representations may represent the examination region in real space, in frequency space, in the projection space, or in any other representation.

The “real space” is the ordinary three-dimensional Euclidean space that corresponds to the space we humans experience with our senses and in which we move. A representation in real space is therefore the more familiar representation for people.

In a representation in real space, also referred to in this description as real-space depiction or real-space representation, the examination region is normally represented by a large number of image elements (pixels or voxels), which may for example be in a raster arrangement in which each image element represents a part of the examination region, wherein each image element may be assigned a colour value or grey value. The colour value or grey value represents a signal intensity, for example the attenuation of X-rays. A format widely used in radiology for storing and processing representations in real space is the DICOM format. DICOM (Digital Imaging and Communications in Medicine) is an open standard for storing and exchanging information in medical image data management.

In a representation in frequency space, also referred to in this description as frequency-space depiction or frequency-space representation, the examination region is represented by a superposition of fundamental vibrations. For example, the examination region may be represented by a sum of sine and/or cosine functions having different amplitudes, frequencies and phases. The amplitudes and phases may be plotted as a function of the frequencies, for example, in a two- or three-dimensional representation. Normally, the lowest frequency (origin) is placed in the centre. The further away from this centre, the higher the frequencies. Each frequency can be assigned an amplitude representing the frequency in the frequency-space depiction and a phase indicating the extent to which the respective vibration is shifted towards a sine or cosine vibration.

A representation in real space can for example be converted (transformed) by a Fourier transform into a representation in frequency space. Conversely, a representation in frequency space can for example be converted (transformed) by an inverse Fourier transform into a representation in real space.

Details about real-space depictions and frequency-space depictions and their respective interconversion are described in numerous publications, see for example https:/see.stanford.edu/materials/lsoftaee261/book-fall-07.pdf.

A representation of an examination region in the projection space is the result of a computed tomography examination prior to image reconstruction. A projection-space depiction can be understood as meaning raw data in the computed tomography examination. In computed tomography, the intensity or attenuation of X-radiation as it passes through the examination object is measured. From this, projection values can be calculated. In a second step, the object information encoded by the projection is transformed into an image (real-space depiction) through a computer-aided reconstruction. The reconstruction can be effected with the Radon transform. The Radon transform describes the link between the unknown examination object and its associated projections.

Details about the transformation of projection data into a real-space depiction are described in numerous publications, see for example K. Fang: The Radon Transformation and Its Application in Tomography, Journal of Physics Conference Series 1903(1):012066.

The at least two representations are the result of a computed tomography examination of the examination region at different X-ray energies. In other words, there are at least two representations generated at different X-ray photon energies, a first representation and a second representation.

The terms X-ray energies and photon energies should not necessarily be understood as meaning exact energy values; they can also be energy ranges. In other words, the X-rays used may be monochromatic or polychromatic. There are however at least two representations of the examination region, in which the energies or energy ranges of the X-rays differ.

One of the at least two representations may be a representation acquired at a lower X-ray energy than the other. In this disclosure, the representation acquired at a lower X-ray energy is referred to as the first representation or as the low-energy representation; the other representation acquired at a higher X-ray energy than the first representation is referred to in this disclosure as the second representation, or high-energy representation. These assignments and designations are arbitrary and serve only to distinguish between the representations.

The at least two representations may for example be the result of a dual-energy computed tomography (DECT).

In DECT, two CT image sets are generated: one based on low-energy X-ray photons and the other based on high-energy photons. As a rule, the technical capabilities of the CT scanner are made use of here, i.e. the maximum and minimum tube voltages are used (for example 140 kVp and 80 kVp). In addition, it is possible with some CT devices to modify the average energy of the tube spectrum through filters. As an alternative to the use of different X-ray spectra, it is also possible for the X-ray photon energies to be separated in the detector, for example by what are known as sandwich detectors.

Different materials or tissues exhibit different absorption behaviour at different X-ray energies. This allows materials in CT images to be differentiated from one another. This effect can for example be exploited in order to perform a material decomposition (see for example Q. Yang et al.: Material decomposition with dual energy CT, 41st Annual Northeast Biomedical Engineering Conference (NEBEC), Troy, NY, USA, 2015, pp. 1-2; DE102017207125A1), which means it is possible to determine the proportion of the signal intensities originating from a particular material (for example the contrast agent or bone tissue or another tissue).

In the present case, the effect is exploited in order to generate a representation of the contrast agent signals. Such a representation of the contrast agent signals represents the signal intensity distribution in the examination region brought about preferably exclusively by the contrast agent.

The X-ray energies in the at least two representations of the examination region are preferably selected such that the difference in signal intensities brought about by contrast agent in the at least two representations is as large as possible.

For example, the absorption spectrum of iodine-containing contrast agents shows an absorption edge (K-edge of iodine) at an X-ray energy of about 33.17 keV; in this case, the absorption capacity of the iodine-containing contrast agent for X-radiation is comparatively large. At an X-ray energy above 125 keV, the absorption capacity of the iodine-containing contrast agent is comparatively low (see for example: I. Danad et al.: Recent Advances in Cardiac Computed Tomography: Dual Energy, Spectral and Molecular CT Imaging, JACC Cardiovasc Imaging, 2015, 8(6): 710-723.

The difference in the signal intensities brought about by iodine-containing contrast agent in the at least two representations is therefore, for example, comparatively large when the first representation (low-energy representation) is acquired at an (average) X-ray energy of about 33.17 keV and the second representation (high-energy representation) at an (average) X-ray energy of about 125 keV.

For example, the absorption spectrum of gadolinium-containing contrast agents shows an absorption edge (K-edge of gadolinium) at an X-ray energy of about 50.2 keV; in this case, the absorption capacity of the gadolinium-containing contrast agent for X-radiation is comparatively large. At an X-ray energy of e.g. 150 keV, the absorption capacity of the gadolinium-containing contrast agent is comparatively low (see for example: I. Danad et al.: Recent Advances in Cardiac Computed Tomography: Dual Energy, Spectral and Molecular CT Imaging, JACC Cardiovasc Imaging, 2015, 8(6): 710-723.

The difference in the signal intensities brought about by gadolinium-containing contrast agent in the at least two representations is therefore, for example, comparatively large when the first representation (low-energy representation) is acquired at an (average) X-ray energy of about 50.2 keV and the second representation (high-energy representation) at an (average) X-ray energy of up to 150 keV.

Similarly to DECT, spectral CT also makes use of the energy-dependent attenuation of X-ray photons by means of photon-counting detectors (T. Flohr et al.: Photon-counting CT review, Phys Med. 2020, 79:126-136). Unlike DECT, which is carried out with only two photon energy levels, spectral CT makes use of multiple energy levels. The simultaneous detection of multiple photon energy levels permits a more differentiated characterization of tissues based on the K-edge behaviour of multiple materials (see for example A. So, S. Nicolaou: Spectral Computed Tomography: Fundamental Principles and Recent Developments, Korean Journal of Radiology, 2020, 21, 10.3348/kjr.2020.0144).

The representation of the contrast agent signals can for example be generated at least partially by subtracting a representation having higher signal intensities brought about by contrast agent from a representation having lower signal intensities brought about by contrast agent.

Assuming that the signal intensities in representations of regions of the examination region in which no contrast agent is present do not change as a result of the change in X-ray energy or change only slightly, then the subtraction of a high-energy representation from a low-energy representation in real space results, for example, in a representation of the examination region in which the signal intensities are essentially determined by the contrast agent (since the signal intensities of the remaining regions are minimized by subtraction).

This representation of the contrast agent signals may be added α times to one of the at least two representations to generate a synthetic representation of the examination region of the present disclosure, where α is a negative or positive real number.

If the representation of the contrast agent signals had been generated by subtracting the high-energy representation from the low-energy representation, then a single (α=1) addition of the representation of the contrast agent signals to the high-energy representation gives rise again to the low-energy representation. A single (α=1) addition of the representation of the contrast agent signals to the low-energy representation gives rise to a representation of the examination region in which the signals brought about by contrast agent in the examination region have higher intensities than in the low-energy representation. With a values greater than 1, a further increase in signal intensity can be achieved. With a values of less than 1, the signal intensities brought about by contrast agent can be reduced.

However, the signal intensities for many tissue types are dependent on the X-ray energy. For water and tissue types having a high water content, the dependence of the signal intensities on the X-ray energy can be disregarded, but for fat (for example fatty tissue) and/or bone tissue the dependence can be so great that it should not be disregarded.

Thus, in one embodiment, regions in which the signal intensities originate from bone tissue and/or adipose tissue and/or air are excluded from the described differential enhancement.

In a first step, regions in the high-energy representation and/or in the low-energy representation that represent bone tissue, adipose tissue and/or air can be identified. This can be done for example on the basis of the signal intensities that are typical for tissue types. It is also possible to use material decomposition to identify bone tissue, adipose tissue and/or air (see for example: R. Bhayana et al.: Material decomposition with dual- and multi-energy computed tomography, MRS Communications, 2020, 10(4), 558-565).

In a second step, a first synthetic representation of the examination region can be generated in which bone tissue, adipose tissue and/or air are represented as per the high-energy representation (i.e. with the lowest possible signal intensity) and in which the other tissues (for example muscles, blood vessels) and also regions in which contrast agent is present are represented as per the low-energy representation (i.e. with the highest possible signal intensity).

In a fourth step, the high-energy representation can be subtracted from the first synthetic representation. The result is a representation of the contrast agent signals.

In a fifth step, the representation of the contrast agent signals can be added α times to the high-energy representation and/or to the low-energy representation, where α is a negative or positive real number. Preferably, the representation of the contrast agent signals is added α times to the high-energy representation, since this usually has a higher signal-to-noise ratio than the low-energy representation. The result is a second synthetic representation of the examination region of the examination object. The second synthetic representation may be output, stored and/or transmitted to a separate computer system.

It is also possible to add a representation of the contrast agent signals a times to a virtual non-contrast agent representation (VNC representation), where α is a negative or positive real number. A VNC representation is a synthetic representation of the examination region in which the signal intensities brought about by contrast agent are eliminated by post-processing, with the result that such a representation gives the impression that no contrast agent had been administered. The technique for generating VNC representations also referred to as virtual non-contrast imaging is a widely used technique that is employed inter alia when no representation of the examination region without contrast agent (true non-contrast (TNC) image) is available, but such a representation is required for the examination (see for example: H. Scheffel et al.: Dual-Energy Contrast-Enhanced Computed Tomography for the Detection of Urinary Stone Disease, Investigative Radiology 2007, 42(12): pp. 823-829; M. K. Virarkar et al.: Virtual Non-contrast Imaging in The Abdomen and The Pelvis: An Overview, Seminars in Ultrasound, CT and MRI, volume 43, issue 4, 2022, pages 293-310).

It is also possible for one of the at least two received representations to be a VNC representation, for example the high-energy representation.

It is also possible for one or more of the at least two received representations to be virtual monoenergetic CT images (VMI representations). VMI (virtual monoenergetic imaging) permits the reconstruction of DECT data sets or spectral CT data sets (photon counting CT) at a selected hypothetical energy level that would result from an image with a true monoenergetic X-ray beam. Depending on the DECT system, such a VMI representation can be generated either in the projection space or in real space. The basic principle of VMI is based on (DECT) material decomposition. In particular, DECT makes it possible to quantify two base materials, which permits reconstruction of the proportion of the total mass of each material in each voxel. Based on this material-specific information, it is possible to extrapolate the density of each voxel from the DECT data set to a particular energy level within a range of 40-200 kiloelectronvolts (keV). The generation of VMI representations is described in the literature (see for example: D. Cester et al.: Virtual monoenergetic images from dual-energy CT: systematic assessment of task-based image quality performance, Quant Imaging Med Surg 2022, 12(1): 726-741.

The at least two received representations may, as described, be representations of the examination region in real space, in frequency space or in the projection space.

It is accordingly possible to carry out the operations described in this description for generating a representation of the contrast agent signals and a (first and/or second) synthetic representation in real space, in frequency space and/or in the projection space.

In one embodiment, the representation of the contrast agent signals is generated in frequency space.

The frequency-space representation of the contrast agent signals may be subjected to filtering and/or noise suppression and/or another operation before being added α times to a frequency-space high-energy representation and/or to a frequency-space low-energy representation and/or to a frequency-space VNC representation.

For example, a weight function may be applied to the frequency-space representation of the contrast agent signals that, for example, weights lower frequencies more highly than higher frequencies. In a frequency-space depiction, contrast information is encoded in the low-frequency range, whereas fine-structure information is encoded in the higher-frequency range. These statements on contrast information and on fine-structure information relate here to the corresponding real-space depictions. Image noise is typically evenly distributed in the frequency depiction. Such a frequency-dependent weight function has the effect of a filter. The filter increases the signal-to-noise ratio by reducing the spectral noise density for high frequencies.

The gain factor α may be a fixed value or it may be specified by a user (for example entered into the computer system or selected from a virtual menu) and/or determined in an automated manner. It is for example possible to define at least one region in a real-space depiction of the low-energy representation and/or of the high-energy representation and/or to have it selected by a user and to set the gain factor such that a grey value in the synthetically contrast-enhanced region in the real-space depiction assumes a defined value and/or is above or below a threshold value and/or that two grey values in two different selected or defined regions are at a defined distance from one another and/or are at a distance from one another that is above or below a threshold value. It is also possible to employ other criteria in the automated determination of the gain factor α. The criteria for the automated determination of the gain factor may for example be based on the histogram of a low-energy representation and/or high-energy representation and/or synthetic representation. Such a histogram may show the number of pixels/voxels having a defined tone value or grey value.

The gain value a may be for example a value between 0 and 1. The gain value a may be for example a value between −1 and 0. The gain value a may be for example a value between −10 and −1. The gain value a may be for example a value between 1 and 10. The gain factor α may however also be smaller than −10 or it may be greater than 10.

The invention will be more particularly elucidated hereinbelow with reference to drawings, without any intention to restrict the invention to the features or combinations of features shown in the drawings. Statements made in the description of the drawings are intended to be applicable generally and not just to the examples shown in the drawings.

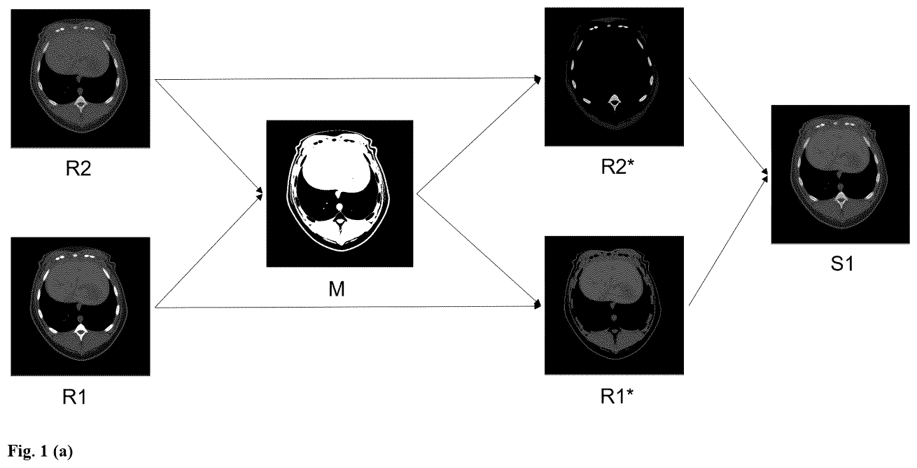

FIGS. 1A and 1B show in schematic form an embodiment for the generation of a synthetic contrast-enhanced CT image.

The representations R1, R1*, R2, R2*, R2^{F}, M, S1, S1F, KRF, KR^{F,w}, S2^{F} and S2 shown in FIGS. 1A and/or 1B represent an examination region of a pig after the weight-adapted administration of gadoxetate disodium, where the examination region comprises the pig's liver.

The starting point of the method shown in FIGS. 1A and 1B is a first representation R1 and a second representation R2. Both the first representation R1 and the second representation R2 represent the examination region of the examination object in real space.

The representations R1 and R2 are the result of a computed tomography examination at different X-ray energies. The first representation R1 was acquired at a lower X-ray energy than the second representation R2. The first representation R1 is thus the low-energy representation, whereas the second representation R2 is the high-energy representation.

On the basis of representation R1 and/or representation R2, regions in representation R1 and/or representation R2 are identified that represent bone tissue, adipose tissue and air. In the present case, pixels/voxels having signal intensities in the representation R1 and in the representation R2 of less than −10 HU or greater than 150 HU were ascribed to bone tissue, adipose tissue or air. A mask M was generated in which the grey values of the pixels/voxels representing bone tissue, adipose tissue or air are set to zero (black). The remaining regions represent contrast agent, muscle tissue, liver tissue or other predominantly water-containing tissue. The grey values of the pixels/voxels of the remaining regions were set to 1 (white) in the mask M.

The mask M can be used to generate a first synthetic representation S1 of the examination region. If the mask M is multiplied by the first representation R1 (pixel-/voxelwise multiplication of the grey values of the first representation R1 by the values of the mask M), a modified representation R1* is generated in which the grey values of the regions representing bone tissue, adipose tissue or air are set to zero (black). The modified representation R1* thus shows only contrast agent, muscle tissue, liver tissue and other predominantly water-containing tissue in a contrasted form.

If a mask inverse to the mask M (in which the grey values of the pixels/voxels representing bone tissue, adipose tissue or air are set to 1 (white) and the grey values of the remaining pixels/voxels are set to zero (black)) is multiplied by the second representation R2, this results in a modified second representation R2* in which only regions representing bone tissue, adipose tissue or air are displayed (contrasted); the remaining regions are black.

On the basis of the modified first representation R1* and the modified second representation R2*, the first synthetic representation S1 is generated. The first synthetic representation S1 is a combination of the modified first representation R1* and the modified second representation R2*. It can be generated through addition of the modified first representation R1* and the modified second representation R2*. In the first synthetic representation S1, regions representing bone tissue, adipose tissue or air are displayed as per the modified first representation R1* (and as per the first representation R1) and the remaining regions as per the modified second representation R2* (and as per the second representation R2). In other words, the first synthetic representation S1 consists of those pixels/voxels that in the first modified representation R1* (and in the first representation R1) represent bone tissue, adipose tissue, or air and those pixels/voxels that in the second modified representation R2* (and in the second representation) represent neither bone tissue, adipose tissue, nor air.

On the basis of the second representation R2 and the first synthetic representation S1, a representation of the contrast agent signals can be generated in a further step. The representation of the contrast agent signals can be generated by subtracting the second representation R2 from the first synthetic representation S1. Since the grey values of corresponding pixels/voxels of the second representation R2 and of the first synthetic representation S1 are in each case the same for regions representing bone tissue, adipose tissue or air, the subtraction results in these regions being displayed as black in the representation of the contrast agent signals. In the regions displaying contrast agent, muscle tissue, liver tissue and other predominantly water-containing tissues, the subtraction has the effect of minimizing the signal intensities originating from pure muscle tissues, liver tissues and other predominantly water-containing tissues. In the case of regions in which contrast agent is present, the subtraction results in a slight reduction in signal intensities. The subtraction thus leaves behind a representation of the examination region in the form of the representation of the contrast agent signals in which the signal intensity distribution is brought about almost exclusively by the contrast agent. This representation of the contrast agent signals can be added α times to the first and/or second representation and/or to a VNC representation of the examination region.

However, in the embodiment shown in FIG. 1B, the subtraction takes place not in real space but in frequency space. For this, the real-space depictions are first converted into frequency-space depictions. The second real-space representation R2 can be converted (transformed) into a second frequency-space representation R2^{F} by a Fourier transform operation. The first synthetic real-space representation S1 can be converted (transformed) into a first synthetic frequency-space representation S1^{F} by a Fourier transform operation.

On the basis of the second frequency-space representation R2^{F} and the first synthetic frequency-space representation S1^{F}, a representation of the contrast agent signals KRF is generated in frequency space. For example, the representation of the contrast agent signals KRF in frequency space can be generated by subtracting the second frequency-space representation R2^{F} from the first synthetic frequency-space representation S1^{F}.

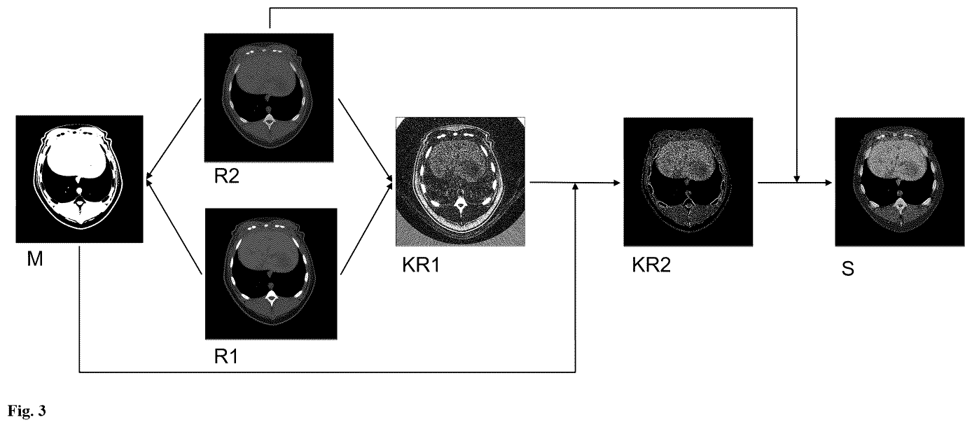

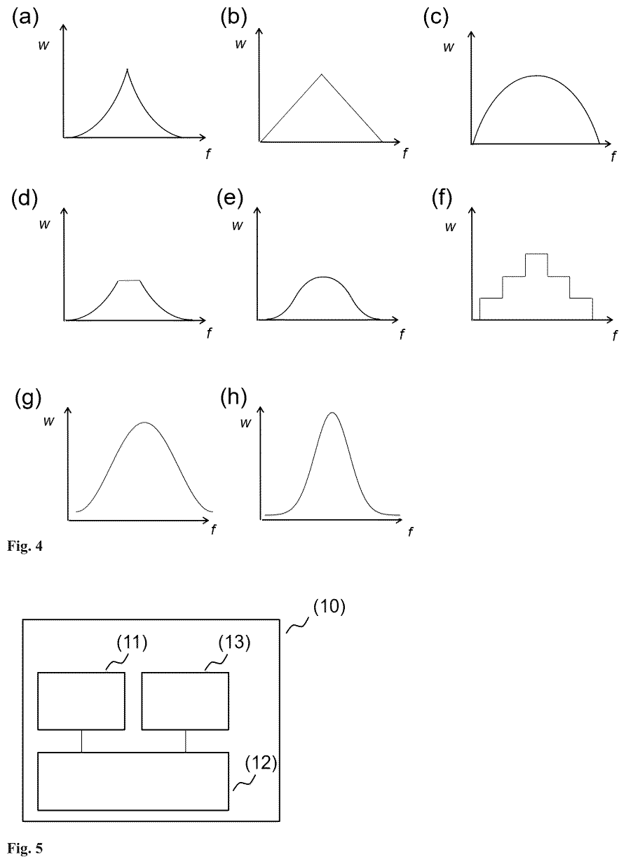

In the embodiment shown in FIG. 1B, a weight function is applied to the representation of the contrast agent signals KR^{F} in frequency space. Such a weight function can be used for example to increase the signal-to-noise ratio. Examples of weight functions are shown in FIG. 4.

The result of the frequency-dependent weighting of the representation of the contrast agent signals KR^{F} in frequency space is a weighted representation of the contrast agent signals KR^{F,w} in frequency space. In the weighting, the amplitude values of the individual frequencies of the representation of the contrast agent signals KR^{F} are multiplied by frequency-dependent weight factors w (f=frequency).

It should be noted that the weighting is an optional step.

On the basis of the (optionally) weighted representation of the contrast agent signals KR^{F,w} in frequency space and of the second frequency-space representation R2^{F}, a second synthetic frequency-space representation S2F is generated. In the present example, the second synthetic frequency-space representation S2F is generated by adding the (optionally) weighted representation of the contrast agent signals KR^{F,w} in frequency space a times to the second frequency-space representation R2^{F}, where a is a positive or negative real number. In the present example, α is >1, i.e. the signal intensity distribution brought about by contrast agent in the examination region is increased in the second synthetic representation S2 compared to the second frequency-space representation R2.

The second synthetic representation S2 can be generated from the second synthetic frequency-space representation S2F by an inverse Fourier transform.

The second synthetic representation S2 can be output, i.e. displayed on a monitor and/or printed by means of a printer, stored in a data memory and/or transmitted to a separate computer system.

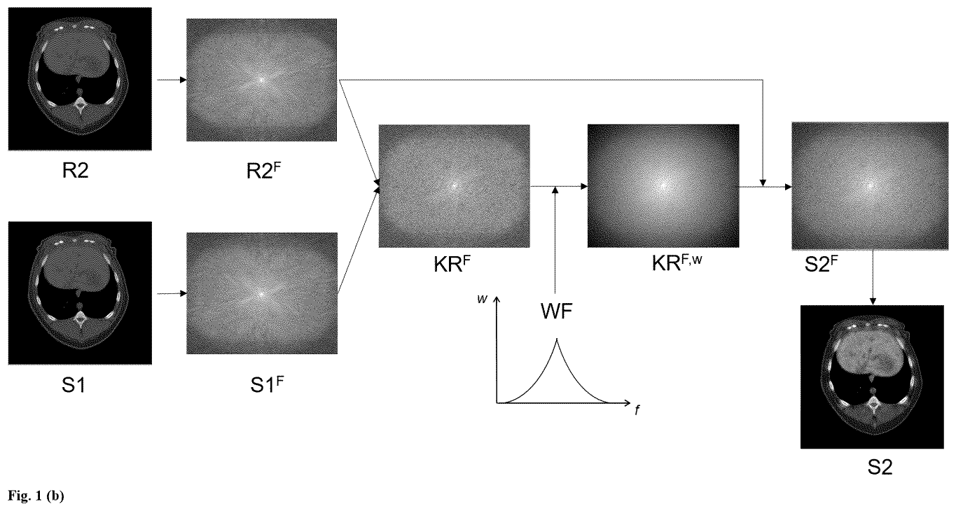

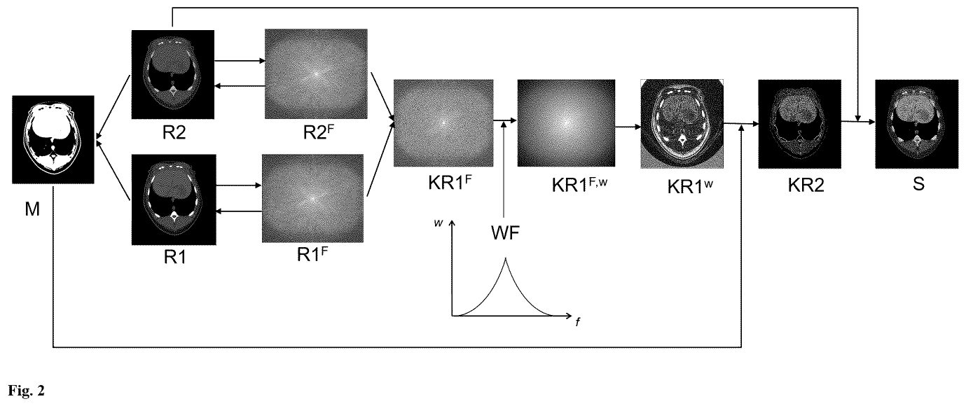

FIG. 2 shows in schematic form a further embodiment for the generation of a synthetic contrast-enhanced CT image. The embodiment shown in FIG. 2 is a variant of the embodiment shown in FIGS. 1A and 1B.

The representations M, R1, R1^{F}, R2, R2^{F}, KR1^{F}, KR1^{F,w}, KR1^{w}, KR2 and S shown in FIG. 2 represent an examination region of a pig after the weight-adapted administration of gadoxetate disodium, where the examination region comprises the pig's liver.

The starting point of the method shown in FIG. 2 can (as in the method shown in FIGS. 1A and 1B) be a first representation R1 and a second representation R2. Both the first representation R1 and the second representation R2 represent the examination region of the examination object in real space.

The starting point of the method can however also be the representation R1^{F} and the representation R2^{F}. Both the representation R1^{F} and the representation R2^{F} represent the examination region of the examination object in frequency space.

The representations R1 and R2 and also R1^{F} and R2^{F} are the result of a computed tomography examination at different X-ray energies. The representation R1/R1^{F} was acquired at a lower X-ray energy than the representation R2/R2^{F}. The representation R1/R1^{F} is thus the low-energy representation, whereas the representation R2/R2^{F} is the high-energy representation.

The representations R1 and R1^{F} are mutually interconvertible, for example the frequency-space representation R1^{F} can be generated from the real-space representation R1 by a Fourier transform and the real-space representation R1 can be generated from the frequency-space representation R1^{F} by an inverse Fourier transform. Similarly, the frequency-space representation R2^{F} can be generated from the real-space representation R2 by a Fourier transform and the real-space representation R2 can be generated from the frequency-space representation R2^{F} by an inverse Fourier transform.

As shown in FIG. 2, a mask M is generated on the basis of the representation R1 and/or the representation R2. The mask M can be generated as described in relation to FIG. 1A. In the mask M, the grey values of the pixels/voxels representing bone tissue, adipose tissue and/or air are set to zero (black) and the grey values of the remaining pixels/voxels are set to 1 (white).

On the basis of the frequency-space representations R1^{F} and R2^{F}, a first representation of the contrast agent signals KR1^{F} can be generated in frequency space. In the present example, the first representation of the contrast agent signals KR1^{F} in frequency space is generated by subtracting the high-energy frequency-space representation R2^{F} from the low-energy frequency-space representation R1^{F}.

On the basis of the first representation of the contrast agent signals KR1^{F} in frequency space, a weighted first representation of the contrast agent signals KR1^{F},w in frequency space is generated by weighting with a weight function WF. Such a weight function can be used for example to increase the signal-to-noise ratio. Examples of weight functions are shown in FIG. 4.

A transform operation, for example an inverse Fourier transform, generates a weighted first representation of the contrast agent signals KR1^{w} in real space from the weighted first representation of the contrast agent signals KR1^{F},w in frequency space.

In a next step, the grey values of all pixels/voxels representing bone tissue, adipose tissue and/or air in the weighted first representation of the contrast agent signals KR1^{w} in real space are set to zero (black). This can be done for example by multiplying the weighted first representation of the contrast agent signals KR1^{w} in real space by the mask M. The result is a second representation of the contrast agent signals KR2 in real space.

In the present example, the second representation of the contrast agent signals KR2 in real space is added α times to the real-space high-energy representation R2. The result is a synthetic representation S of the examination region of the examination object in which, depending on the magnitude of the gain factor α, the contrast between regions containing contrast agent and regions that do not contain contrast agent is increased or decreased.

The synthetic representation S can be output, i.e. displayed on a monitor and/or printed by means of a printer, stored in a data memory and/or transmitted to a separate computer system.

FIG. 3 shows in schematic form a further embodiment for the generation of a synthetic contrast-enhanced CT image.

The representations M, R1, R2, KR1, KR2 and S shown in FIG. 3 represent an examination region of a pig after the weight-adapted administration of gadoxetate disodium, where the examination region comprises the pig's liver.

In the embodiment shown in FIG. 3, all operations are carried out in real space. As in the method shown in FIGS. 1A and 1B, the starting point is a first representation R1 of the examination region of an examination object in real space and a second representation R2 of the examination region of the examination object in real space.

The representations R1 and R2 are the result of a computed tomography examination at different X-ray energies. The representation R1 was acquired at a lower X-ray energy than the representation R2. The representation R1 is thus the low-energy representation, whereas the representation R2 is the high-energy representation.

A mask M is generated on the basis of the representation R1 and/or the representation R2. The mask M can be generated as described in relation to FIG. 1A. In the mask M, the grey values of the pixels/voxels representing bone tissue, adipose tissue and/or air are set to zero (black) and the grey values of the remaining pixels/voxels are set to 1 (white).

On the basis of the representation R1 and/or the representation R2, a first representation of the contrast agent signals KR1 is generated. The representation of the contrast agent signals KR1 can be generated by subtracting the high-energy representation R2 from the low-energy representation R1.

On the basis of the first representation of the contrast agent signals KR1, a second representation of the contrast agent signals KR2 is generated. In the present example, this is done by multiplying the mask M by the first representation of the contrast agent signals KR1. This multiplication sets all grey values representing bone tissue, adipose tissue and/or air in the first representation of the contrast agent signals KR1 to zero (black). The grey values of the remaining pixels/voxels remain unchanged.

In the present example, the second representation of the contrast agent signals KR2 is added α times to the high-energy representation R2. The result is a synthetic representation S of the examination region of the examination object in which, depending on the magnitude of the gain factor α, the contrast between regions containing contrast agent and regions that do not contain contrast agent is increased or decreased.

The synthetic representation S can be output, i.e. displayed on a monitor and/or printed by means of a printer, stored in a data memory and/or transmitted to a separate computer system.

FIGS. 4A-4H show examples of frequency-dependent weight functions that can be used for weighting the representation of the contrast agent signals in frequency space. For simplicity, the weight functions are represented as two-dimensional graphs in which the weight factors w (ordinate) are plotted as a function of the frequency f (abscissa).

FIG. 4A shows a weight function in which the weight factors decrease from the centre exponentially as the frequency increases.

FIG. 4B shows a weight function in which the weight factors decrease from the centre linearly as the frequency increases.

FIG. 4C shows a weight function in which the weight factors decrease from the centre in the form of a reverse parabola as the frequency increases.

FIG. 4D shows a weight function in which the weight factors are constant over a defined range about the centre and then decrease exponentially from a threshold frequency.

FIG. 4E shows a weight function in which the weight factors have the course of a cosine function about the centre.

FIG. 4F shows a weight function in which the weight factors have the course of a step function about the centre.

FIG. 4G shows a weight function in which the weight factors have the course of a Gaussian distribution function about the centre.

FIG. 4H shows a weight function in which the weight factors have the course of a Hann function about the centre.

Combinations of the weight functions shown and further/other weight functions are possible. Examples of other weight functions can be found for example at https://de.wikipedia.org/wiki/Fensterfunktion#Beispiele_von_Fensterfunktionen; F. J. Harris et al.: On the Use of Windows for Harmonic Analysis with the Discrete Fourier Transform, Proceedings of the IEEE, vol. 66, No. 1, 1978; https://docs.scipy.org/doc/scipy/reference/signal.windows.html; K. M. M. Prabhu: Window Functions and Their Applications in Signal Processing, CRC Press, 2014, 978-1-4665-1583-3).

Weight functions that can be used are also referred to in the literature as window functions.

Preference is given to using weighting functions of proven utility for the weighting of k-space data in MR imaging and spectroscopy, for example the Hann function (also referred to as the Hann window, see for example: Hanning window, see for example R. Pohmann et al.: Accurate phosphorus metabolite images of the human heart by 3D acquisition-weighted CSI, Magnetic Resonance in Medicine: An Official Journal of the International Society for Magnetic Resonance in Medicine 45.5 (2001): 817-826).

Another preferred weight function is the Poisson function (Poisson window).

FIG. 5 shows by way of example and in schematic form a computer system according to the present disclosure.

A “computer system” is an electronic data processing system that processes data by means of programmable calculation rules. Such a system typically comprises a “computer”, which is the unit that includes a processor for carrying out logic operations, and peripherals. In computer technology, “peripherals” denotes all devices that are connected to the computer and are used to control the computer and/or are used as input and output devices. Examples thereof are monitor (screen), printer, scanner, mouse, keyboard, drives, camera, microphone, speakers, etc. Internal ports and expansion cards are also regarded as peripherals in computer technology.

The computer system (1) shown in FIG. 5 comprises an input unit (11), a control and calculation unit (12) and an output unit (13).

The control and calculation unit (12) serves for control of the computer system (1), for coordination of the data flows between the units of the computer system (1), and for the performance of calculations.

The control and calculation unit (12) is configured:
to cause the input unit (11) to receive and/or generate at least two representations of an examination region of an examination object, where the at least two representations represent the examination region after administration of a contrast agent, where the at least two representations are the result of a computed tomography examination of the examination region at different X-ray energies,
to generate a representation of the contrast agent signals on the basis of the at least two received representations, where the representation of the contrast agent signals represents a signal intensity distribution brought about by the contrast agent in the examination region,
to generate a synthetic representation of the examination region, where the generation of the synthetic representation comprises an α-fold addition of the representation of the contrast agent signals to one of the at least two received representations or to a virtual non-contrast agent representation of the examination region, where α is a negative or positive real number,
to cause the output unit (13) to output the synthetic representation and/or to store it and/or to transmit it to a separate computer system.

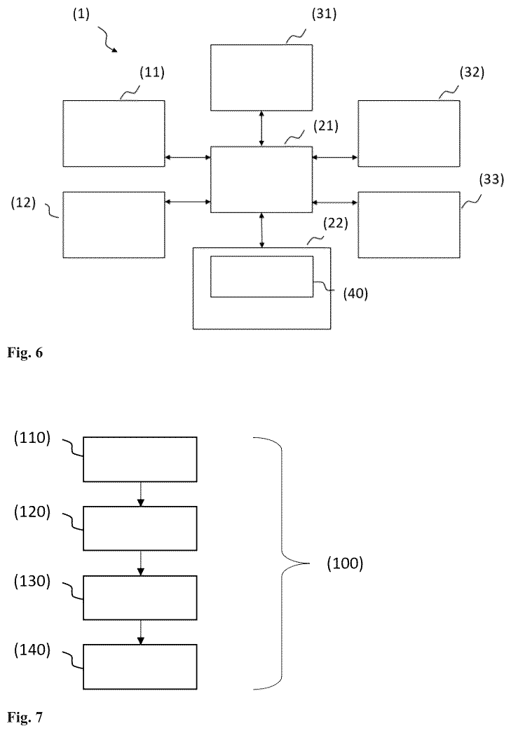

FIG. 6 shows by way of example and in schematic form a further embodiment of the computer system.

The computer system (1) comprises a processing unit (21) connected to a memory (22). The processing unit (21) and the memory (22) form a control and calculation unit, as shown in FIG. 5.

The processing unit (21) may comprise one or more processors alone or in combination with one or more memories. The processing unit (21) may be customary computer hardware that is able to process information such as digital images, computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of the same or of a different computer system.

The memory (22) may be customary computer hardware that is able to store information such as digital images (for example representations of the examination region), data, computer programs and/or other digital information either temporarily and/or permanently. The memory (22) may comprise a volatile and/or non-volatile memory and may be fixed in place or removable. Examples of suitable memories are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (21) may be connected not just to the memory (22), but also to one or more interfaces (11, 12, 31, 32, 33) in order to display, transmit and/or receive information. The interfaces can comprise one or more communication interfaces (32, 33) and/or one or more user interfaces (11, 12, 31). The one or more communication interfaces may be configured to send and/or receive information, for example to and/or from a CT scanner, other computer systems, networks, data memories or the like. The one or more communication interfaces may be configured to transmit and/or receive information via physical (wired) and/or wireless communication connections. The one or more communication interfaces may comprise one or more interfaces for connection to a network, for example using technologies such as mobile telephone, wifi, satellite, cable, DSL, optical fibre and/or the like. In some examples, the one or more communication interfaces may comprise one or more close-range communication interfaces configured to connect devices having close-range communication technologies such as NFC, RFID, Bluetooth, Bluetooth LE, ZigBee, infrared (e.g. IrDA) or the like.

The user interfaces may include a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image- or video-recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces may in addition comprise one or more interfaces for communication with peripherals such as printers and the like.

One or more computer programs (40) may be stored in the memory (22) and executed by the processing unit (21), which is thereby programmed to fulfil the functions described in this description. The retrieving, loading and execution of instructions of the computer program (40) may take place sequentially, such that an instruction is respectively retrieved, loaded and executed. However, the retrieving, loading and/or execution may also take place in parallel.

The computer system of the present disclosure may be designed as a laptop, notebook, netbook and/or tablet PC; it may also be a component of a CT scanner.

The present invention also provides a computer program product. Such a computer program product includes a non-volatile data carrier, for example a CD, a DVD, a USB stick or another data storage medium. Stored on the data carrier is a computer program. The computer program can be loaded into a working memory of a computer system (more particularly into a working memory of a computer system of the present disclosure), where it causes the computer system to execute the following steps:
receiving and/or generating at least two representations of an examination region of an examination object, where the at least two representations represent the examination region after administration of a contrast agent, where the at least two representations are the result of a computed tomography examination of the examination region at different X-ray energies,
generating a representation of the contrast agent signals on the basis of the at least two received representations, where the representation of the contrast agent signals represents a signal intensity distribution brought about by the contrast agent in the examination region,
generating a synthetic representation of the examination region, where the generation of the synthetic representation comprises an α-fold addition of the representation of the contrast agent signals to one of the at least two received representations or to a virtual non-contrast agent representation of the examination region, where α is a negative or positive real number,
outputting and/or storing the synthetic representation and/or transmitting the synthetic representation to a separate computer system.

The computer program product can also be marketed in combination (in a set) with the contrast agent. Such a set is also referred to as a kit. Such a kit comprises the contrast agent and the computer program product. It is also possible for such a kit to comprise the contrast agent and means that allow the purchaser to obtain the computer program, for example by downloading it from a webpage. These means may include a link, i.e. an address of the webpage on which the computer program can be obtained, for example from which the computer program can be downloaded to a computer system connected to the internet. These means may include a code (for example an alphanumeric string or a QR code, or a DataMatrix code or a barcode or another optically and/or electronically readable code) that gives the purchaser access to the computer program. Such a link and/or code may for example be printed on a packaging of the contrast agent and/or on a package leaflet of the contrast agent. A kit is thus a combination product comprising a contrast agent and a computer program (for example in the form of access to the computer program or in the form of executable program code on a data carrier) that are offered for sale together.

FIG. 7 shows by way of example and in schematic form an embodiment of the computer-implemented method for the generation of a synthetic contrast-enhanced CT image. The method (100) comprises the steps of:
(110) receiving and/or generating at least two representations of an examination region of an examination object, where the at least two representations represent the examination region after administration of a contrast agent, where the at least two representations are the result of a computed tomography examination of the examination region at different X-ray energies,
(120) generating a representation of the contrast agent signals on the basis of the at least two received representations, where the representation of the contrast agent signals represents a signal intensity distribution brought about by the contrast agent in the examination region,
(130) generating a synthetic representation of the examination region, where the generation of the synthetic representation comprises an α-fold addition of the representation of the contrast agent signals to one of the at least two received representations or to a virtual non-contrast agent representation of the examination region, where α is a negative or positive real number,
(140) outputting and/or storing the synthetic representation and/or transmitting the synthetic representation to a separate computer system.

1. A computer-implemented method comprising the steps of:
receiving and/or generating at least two representations (R1, R1^{F}, R2, R2^{F}) of an examination region of an examination object, where the at least two representations (R1, R1^{F}, R2, R2^{F}) represent the examination region after administration of a contrast agent, where the at least two representations (R1, R1^{F}, R2, R2^{F}) are the result of a computed tomography examination of the examination region at different X-ray energies,
generating a representation of the contrast agent signals (KR^{F}, KR^{F,w}, KR1^{F}, KR1^{F,w}, KR1, KR2) on the basis of the at least two received representations (R1, R1^{F}, R2, R2^{F}), where the representation of the contrast agent signals (KR^{F}, KR^{F,w}, KR1^{F}, KR1^{F,w}, KR1, KR2) represents a signal intensity distribution brought about by the contrast agent in the examination region,
generating a synthetic representation (S, S2, S2^{F}) of the examination region, where the generation of the synthetic representation (S, S2, S2^{F}) comprises an α-fold addition of the representation of the contrast agent signals (KR^{F}, KR^{F,w}, KR1^{F}, KR1^{F,w}, KR1, KR2) to one of the at least two received representations (R1, R1^{F}, R2, R2^{F}) or to a virtual non-contrast agent representation of the examination region, where α is a negative or positive real number, and
outputting and/or storing the synthetic representation (S, S2, S2^{F}) and/or transmitting the synthetic representation (S, S2, S2^{F}) to a separate computer system.

2. The method of claim 1, wherein the at least two representations (R1, R1^{F}, R2, R2^{F}) comprise a first representation (R1, R1^{F}) and a second representation (R2, R2^{F}), where the first representation (R1, R1^{F}) was acquired at a lower X-ray energy than the second representation (R2, R2^{F}).

3. The method of claim 2, wherein the generation of the representation of the contrast agent signals (KR^{F}, KR^{F,w}, KR1^{F}, KR1^{F,w}, KR1, KR2) comprises the step of:
subtracting the second representation (R2, R2^{F}) from the first representation (R1, R1^{F}).

4. The method of claim 2, wherein the generation of the representation of the contrast agent signals (KRF, KR^{F,w}, KR1^{F}, KR1^{F,w}, KR1, KR2) comprises the step of:
setting the grey values or tone values of those pixels/voxels of the representation of the contrast agent signals (KR^{F}, KR^{F,w}, KR1^{F}, KR1^{F,w}, KR1, KR2) that represent bone tissue, adipose tissue and/or air to zero.

5. The method of claim 2, wherein the generation of the synthetic representation (S, S2, S2^{F}) comprises the step of:
α-fold addition of the representation of the contrast agent signals (KR^{F}, KR^{F,w}, KR1^{F}, KR1^{F,w}, KR1, KR2) to the second representation (R2, R2^{F}) or to the virtual non-contrast agent representation.

6. The method of claim 2, wherein the first representation (R1) and the second representation (R2) are representations of the examination region in real space.

7. The method of claim 2, wherein the first representation (R1^{F}) and the second representation (R2^{F}) are representations of the examination region in frequency space.

8. The method of claim 1, wherein the representation of the contrast agent signals (KR^{F}, KR^{F},w KR1^{F}, KR1^{F},w) is a representation of the examination region in frequency space, the method further comprising the step of:
weighting the representation of the contrast agent signals (KR^{F}, KR^{F,w}, KR1^{F}, KR1^{F,w}) in frequency space with a frequency-dependent weight function (WF).

9. The method of claim 1, wherein a is greater than 1.

10. The method of claim 1, wherein a is greater than zero and less than 1.

11. The method of claim 1, wherein a is less than zero.

12. The method of claim 1, further comprising the steps of:
receiving the at least two representations (R1, R2) of the examination region of the examination object, where the at least two representations (R1, R2) comprise a first representation (R1) and a second representation (R2), where the first representation (R1) was acquired at a lower X-ray energy than the second representation (R2), where the first representation (R1) and the second representation (R2) represent the examination region in real space after administration of the contrast agent,
transforming the first representation (R1) in real space into a first representation (R1^{F}) in frequency space,
transforming the second representation (R2) in real space into a second representation (R2^{F}) in frequency space,
generating a representation of the contrast agent signals (KR1^{F}) in frequency space by subtracting the second representation (R2^{F}) in frequency space from the first representation (R1^{F}) in frequency space,
optionally: weighting the representation of the contrast agent signals (KR1^{F}) in frequency space with a frequency-dependent weight function (WF),
generating a first real-space representation of the contrast agent signals (KR1^{w}) by transforming the optionally weighted representation of the contrast agent signals (KR1^{F}, KR1^{F,}w) in frequency space,
generating a second real-space representation of the contrast agent signals (KR2) by setting the grey values or tone values of those pixels/voxels of the first real-space representation of the contrast agent signals (KR1^{w}) that represent bone tissue, adipose tissue and/or air to zero,
generating the synthetic representation (S) of the examination region, where the generation of the synthetic representation (S) comprises an α-fold addition of the second real-space representation of the contrast agent signals (KR2) to the first representation (R1) in real space, to the second representation (R2) in real space or to the virtual non-contrast agent representation of the examination region, and
outputting and/or storing the synthetic representation (S) and/or transmitting the synthetic representation (S) to a separate computer system.

13. The method of claim 1, further comprising the steps of:
receiving and/or generating at least two representations (R1, R2) of an examination region of an examination object, where the at least two representations (R1, R2) comprise a first representation (R1) and a second representation (R2), where the first representation (R1) was acquired at a lower X-ray energy than the second representation (R2), where the first representation (R1) and the second representation (R2) represent the examination region in real space after administration of the contrast agent,
generating a first synthetic representation (S1) on the basis of the first representation (R1) and the second representation (R2), wherein regions in the first synthetic representation (S1) that represent bone tissue, adipose tissue and/or air are depicted such that the corresponding regions in the first representation (R1) and regions in the first synthetic representation (S1) that do not represent bone tissue, adipose tissue or air are depicted such that the corresponding regions in the second representation (R2),
generating the representation of the contrast agent signals (KR1, KR1^{F}, KRF), where the generation of the contrast agent representation comprises the step of: subtracting the second representation (R2, R2^{F}) from the first synthetic representation (S1, S1^{F}) in real space or in frequency space,
optionally: weighting the representation of the contrast agent signals (KR1, KR1^{F}, KRF) with a frequency-dependent weight function (WF),
generating a second synthetic representation (S2, S2^{F}) of the examination region, where the generation of the second synthetic representation (S2, S2^{F}) comprises an α-fold addition of the optionally weighted representation of the contrast agent signals (KR1, KR1^{w}, KR1^{F}, KR1^{F,w}, KR^{F}, KR^{F,w}) to the second representation (R2, R2^{F}) or to the virtual non-contrast agent representation of the examination region in real space or in frequency space, and
outputting and/or storing the second synthetic representation (S2, S2^{F}) and/or transmitting the second synthetic representation (S2, S2^{F}) to a separate computer system.

14. The method of claim 1, wherein the contrast agent is an MRI contrast agent, preferably an intracellular MRI contrast agent, even more preferably a hepatobiliary MRI contrast agent.

15. The method of claim 1, wherein the contrast agent comprises one or more contrast agents selected from the following list:
gadoxetate disodium,
gadolinium(III) 2-[4,7,10-tris(carboxymethyl)-1,4,7,10-tetrazacyclododec-1-yl]acetic acid, gadolinium(III) ethoxybenzyldiethylenetriaminepentaacetic acid,
gadolinium(III) 2-[3,9-bis[1-carboxylato-4-(2,3-dihydroxypropylamino)-4-oxobutyl]-3,6,9,15-tetrazabicyclo [9.3.1]pentadeca-1(15),11,13-trien-6-yl]-5-(2,3-dihydroxypropylamino)-5-oxopentanoate,
dihydrogen [(±)-4-carboxy-5,8,11-tris(carboxymethyl)-1-phenyl-2-oxa-5,8,11-triazatridecan-13-oato(5-)]gadolinate(2-),
tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate, gadolinium 2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate,
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate),
gadolinium 2,2′,2″-{10-1[(1S)-4-(4-butoxyphenyl)-1-carboxybutyl]-1,4,7,10-tetraazacyclododecane-1,4,7-triyl}triacetate,
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium-2,2′,2″-[10-(carboxymethyl)-2-(4-ethoxybenzyl)-1,4,7,10-tetraazacyclododecane-1,4,7-triyl]triacetate,
gadolinium(III) 5,8-bis(carboxylatomethyl)-2-[2-(methylamino)-2-oxoethyl]-10-oxo-2,5,8,11-tetraazadodecane-1-carboxylate hydrate,
gadolinium(III) 2-[4-(2-hydroxypropyl)-7,10-bis(2-oxido-2-oxoethyl)-1,4,7,10-tetrazacyclododec-1-yl]acetate,
gadolinium(III) 2,2′,2″-(10-((2R,3S)-1,3,4-trihydroxybutan-2-yl)-1,4,7,10-tetraazacyclododecane-1,4,7-triyl)triacetate,
a Gd³⁺ complex of a compound of the formula (I) 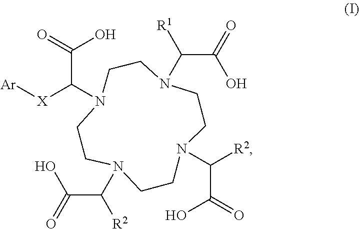
where
Ar is a group selected from 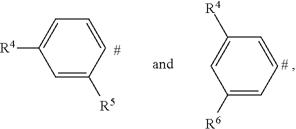
where _{#}is the linkage to X,
X is a group selected from
CH₂, (CH₂)₂, (CH₂)₃, (CH₂)₄ and *—(CH₂)₂O—CH₂—^{#},
where * is the linkage to Ar and ^{#}is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof,
a Gd³⁺ complex of a compound of the formula (II) 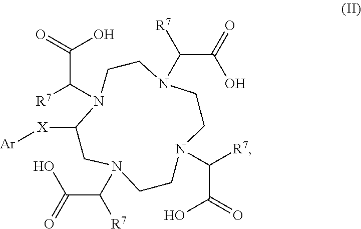
where
Ar is a group selected from 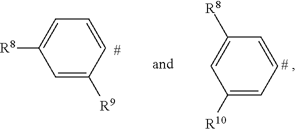
where _{#}is the linkage to X,
X is a group selected from CH₂, (CH₂)₂, (CH₂)₃, (CH₂)₄ and *—(CH₂)₂O—CH₂—^{#}, where * is the linkage to Ar and ^{#}is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

16. A computer system comprising:
a processor; and
a memory that stores an application program configured to perform an operation when executed by the processor, said operation comprising the steps of:
receiving and/or generating at least two representations (R1, R1^{F}, R2, R2^{F}) of an examination region of an examination object, where the at least two representations (R1, R1^{F}, R2, R2^{F}) represent the examination region after administration of a contrast agent, where the at least two representations (R1, R1^{F}, R2, R2^{F}) are the result of a computed tomography examination of the examination region at different X-ray energies,
generating a representation of the contrast agent signals (KR^{F}, KR^{F,w}, KR1^{F}, KR1^{F,w} KR1, KR2) on the basis of the at least two received representations (R1, R1^{F}, R2, R2^{F}), where the representation of the contrast agent signals (KR^{F}, KR^{F,w}, KR1^{F}, KR1^{F,w}, KR1, KR2) represents a signal intensity distribution brought about by the contrast agent in the examination region,
generating a synthetic representation (S, S2, S2^{F} of the examination region, where the generation of the synthetic representation (S, S2, S2^{F}) comprises an α-fold addition of the representation of the contrast agent signals KR^{F}, KR^{F,w}, KR1^{F}, KR1^{F,w}, KR1, KR2) to one of the at least two received representations (R1, R1^{F}, R2, R2^{F}) or to a virtual non-contrast agent representation of the examination region, where α is a negative or positive real number, and
outputting and/or storing the synthetic representation (S, S2, S2^{F}) and/or transmitting the synthetic representation (S, S2, S2^{F}) to a separate computer system.

17. A computer program configured to be loaded into a working memory of a computer system, where the computer program causes the computer system to execute the following steps:
receiving and/or generating at least two representations (R1, R1^{F}, R2, R2^{F}) of an examination region of an examination object, where the at least two representations (R1, R1^{F}, R2, R2^{F}) represent the examination region after administration of a contrast agent, where the at least two representations (R1, R1^{F}, R2, R2^{F}) are the result of a computed tomography examination of the examination region at different X-ray energies,
generating a representation of the contrast agent signals (KR^{F}, KR^{F,w}, KR1^{F}, KR1^{F,w}, KR1, KR2) on the basis of the at least two received representations (R1, R1^{F}, R2, R2^{F}), where the representation of the contrast agent signals (KR^{F}, KR^{F,w}, KR1^{F}, KR1^{F,w}, KR1, KR2) represents a signal intensity distribution brought about by the contrast agent in the examination region,
generating a synthetic representation (S, S2, S2^{F} of the examination region, where the generation of the synthetic representation (S, S2, S2^{F}) comprises an α-fold addition of the representation of the contrast agent signals KR^{F}, KR^{F,w}, KR1^{F}, KR1^{F,w}, KR1, KR2) to one of the at least two received representations (R1, R1^{F}, R2, R2^{F}) or to a virtual non-contrast agent representation of the examination region, where α is a negative or positive real number, and
outputting and/or storing the synthetic representation (S, S2, S2^{F}) and/or transmitting the synthetic representation (S, S2, S2^{F}) to a separate computer system.

18. A method for using a contrast agent in a CT examination method, wherein the CT examination method comprises the steps of:
receiving and/or generating at least two representations (R1, R1^{F}, R2, R2^{F}) of an examination region of an examination object, where the at least two representations (R1, R1^{F}, R2, R2^{F}) represent the examination region after administration of the contrast agent, where the at least two representations (R1, R1^{F}, R2, R2^{F}) are the result of a computed tomography examination of the examination region at different X-ray energies,
generating a representation of the contrast agent signals (KR^{F}, KR^{F,w}, KR1^{F}, KR1^{F,w}, KR1, KR2) on the basis of the at least two received representations (R1, R1^{F}, R2, R2^{F}), where the representation of the contrast agent signals (KR^{F}, KR^{F,w}, KR1^{F}, KR1^{F,w}, KR1, KR2) represents a signal intensity distribution brought about by the contrast agent in the examination region,
generating a synthetic representation (S, S2, S2^{F} of the examination region, where the generation of the synthetic representation (S, S2, S2^{F}) comprises an α-fold addition of the representation of the contrast agent signals KR^{F}, KR^{F,w}, KR1^{F}, KR1^{F,w}, KR1, KR2) to one of the at least two received representations (R1, R1^{F}, R2, R2^{F}) or to a virtual non-contrast agent representation of the examination region, where α is a negative or positive real number, and
outputting and/or storing the synthetic representation (S, S2, S2^{F}) and/or transmitting the synthetic representation (S, S2, S2^{F}) to a separate computer system.

19. A contrast agent for use in a CT examination method, wherein the CT examination method comprises the steps of:
receiving and/or generating at least two representations (R1, R1^{F}, R2, R2^{F}) of an examination region of an examination object, where the at least two representations (R1, R1^{F}, R2, R2^{F}) represent the examination region after administration of the contrast agent, where the at least two representations (R1, R1^{F}, R2, R2^{F}) are the result of a computed tomography examination of the examination region at different X-ray energies,
generating a representation of the contrast agent signals (KR^{F}, KR^{F,w}, KR1^{F}, KR1^{F,w}, KR1, KR2) on the basis of the at least two received representations (R1, R1^{F}, R2, R2^{F}), where the representation of the contrast agent signals (KR^{F}, KR^{F,w}, KR1^{F}, KR1^{F,w}, KR1, KR2) represents a signal intensity distribution brought about by the contrast agent in the examination region,
generating a synthetic representation (S, S2, S2^{F} of the examination region, where the generation of the synthetic representation (S, S2, S2^{F}) comprises an α-fold addition of the representation of the contrast agent signals KR^{F}, KR^{F,w}, KR1^{F}, KR1^{F,w}, KR1, KR2) to one of the at least two received representations (R1, R1^{F}, R2, R2^{F}) or to a virtual non-contrast agent representation of the examination region, where α is a negative or positive real number, and
outputting and/or storing the synthetic representation (S, S2, S2^{F}) and/or transmitting the synthetic representation (S, S2, S2^{F}) to a separate computer system.

20. A kit comprising a contrast agent and a non-volatile storage medium comprising a computer program that can be loaded into a working memory of a computer system, where it causes the computer system to execute the following steps:
receiving and/or generating at least two representations (R1, R1^{F}, R2, R2^{F}) of an examination region of an examination object, where the at least two representations (R1, R1^{F}, R2, R2^{F}) represent the examination region after administration of the contrast agent, where the at least two representations (R1, R1^{F}, R2, R2^{F}) are the result of a computed tomography examination of the examination region at different X-ray energies,
generating a representation of the contrast agent signals (KR^{F}, KR^{F,w}, KR1^{F}, KR1^{F,w}, KR1, KR2) on the basis of the at least two received representations (R1, R1^{F}, R2, R2^{F}), where the representation of the contrast agent signals (KRF, KR^{F,w}, KR1^{F}, KR1^{F,w}, KR1, KR2) represents a signal intensity distribution brought about by the contrast agent in the examination region,
generating a synthetic representation (S, S2, S2^{F} of the examination region, where the generation of the synthetic representation (S, S2, S2^{F}) comprises an α-fold addition of the representation of the contrast agent signals KR^{F}, KR^{F,w}, KR1^{F}, KR1^{F,w}, KR1, KR2) to one of the at least two received representations (R1, R1^{F}, R2, R2^{F}) or to a virtual non-contrast agent representation of the examination region, where α is a negative or positive real number, and
outputting and/or storing the synthetic representation (S, S2, S2^{F}) and/or transmitting the synthetic representation (S, S2, S2^{F}) to a separate computer system.
